(19) United States
(12) Patent Application Publication
(10) Pub. No.: US 2024/0181398 A1
(43) Pub. Date: Jun. 6, 2024

(54) VIRAL FILTER AND METHOD OF VIRAL FILTRATION

(71) Applicant: Global Life Sciences Solutions Operations UK Ltd., Sheffield (GB)

(72) Inventors: Claudia Trindade Nunes, Cardiff South Glamorgan (GB); Martina Lorenzetti, Buccinasco (IT)

(21) Appl. No.: 18/551,012

(22) PCT Filed: Mar. 17, 2022
(86) PCT No.: PCT/EP2022/056989

§ 371 Date: Sep. 18, 2023

Related U.S. Application Data
(60) Provisional application No. 63/163,443, filed on Mar. 19, 2021.
(60) Provisional application No. 63/163,997, filed on Mar. 22, 2021.

(51) Int. Cl. B01D 63/08; B01D 69/02; B01D 69/12; G01N 33/543

(52) U.S. Cl. CPC B01D 63/088; B01D 63/087; B01D 69/02; B01D 69/1216; G01N 33/54388; B01D 2325/021; B01D 2325/02834

(57) ABSTRACT

The present invention provides for simple and rapid filtering of biological samples, whereby a sample can be analyzed in the same device or a different device. In one preferred example, a membrane filter 12 that is particularly useful for the filtration of samples comprising viruses along with other biological materials that need be separated from the viruses is used. A lateral flow device 10 incorporating a filter membrane/membrane filter 12 is also disclosed.

### BACKGROUND

With the advent of point of care (POC) testing, it has become increasingly important to develop diagnostic products which are simple, rapid and convenient for the user to perform. This need has arisen because health care workers need to perform tests in the field outside of a traditional healthcare setting and require results rapidly with a minimum of time given to the performance of a diagnostic test. Providing a diagnostic result in minutes allows a healthcare working to identify disease such as viral disease and to treat a patient as soon as possible.

Point of care diagnostic tests frequently are performed on biological samples, such as whole blood or urine. Cells and particulate matter in biological samples can interfere with fluid flow in a test device, and thus impair the measurement of analytes in the biological fluid.

For example, in blood, red blood cells can interfere with spectroscopic measurements, and as the hematocrit varies, the volume of plasma in a given volume of blood varies. To overcome these problems, red blood cells are separated from plasma to allow for a more defined and uniform sample. The same is true of various components of saliva or urine.

Thus, a device to filter out cells, particulate matter, or debris from a biological sample can improve the quality of an analytical procedure performed on the sample.

The filtration, separation, and isolation of viruses, for example, are critical issues for controlling blood-borne viral infections and for viral research. Membrane-based technology has been identified as a useful method for the separation of biomaterials including viruses, owing to its efficiency, ease of implementation, and cost effectiveness.

### SUMMARY OF THE INVENTION

The present invention provides for simple and rapid filtering of biological samples, whereby a sample can be analyzed in the same device or a different device. In one preferred example, a membrane filter that is particularly useful for the filtration of samples comprising viruses along with other biological materials that need be separated from the viruses is used.

### BRIEF DESCRIPTION OF DRAWINGS

FIG. 1 shows a lateral flow device in accordance with various embodiments of the present invention; and

FIG. 2 shows various component configurations/materials that may be used in lateral flow devices as shown in FIG. 1.

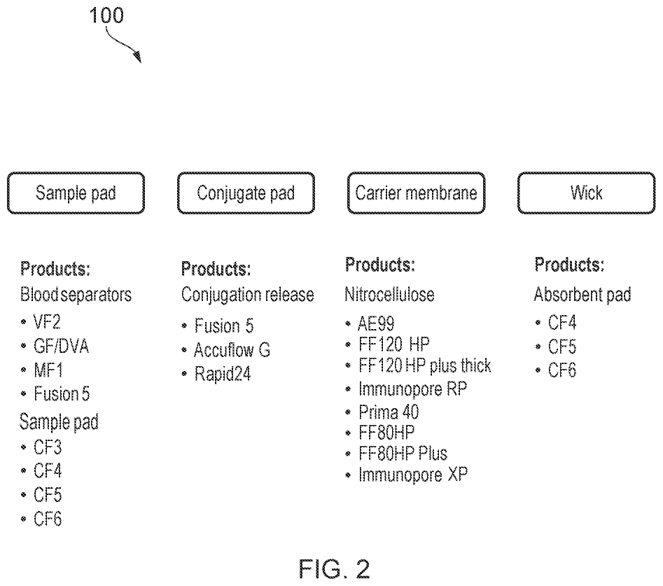

### DETAILED DESCRIPTION

### Definitions

As used herein, a “micropore” refers to an opening, orifice, gap, conduit, passage, chamber, or groove in a membrane/layer, where the micropore or microchannel is of sufficient dimension that allows passage or analysis of at least a single target agent (e.g., a cell, bacteria, virus, biological particle, microbe, or the like). A micropore can allow passage or admit more than one target agent. As used herein, “micro” generally refers to micrometer scale dimensions.

As used herein, a “nanopore” refers to an opening, orifice, gap, conduit, passage, chamber, or groove in a membrane/layer, where the nanopore or nanochannel is of dimension or configuration that prevents passage of a single target agent. As used herein, “nano” generally refers to nanometer scale dimensions.

As used herein, “pore size” generally refers to the width of a micropore or nanopore, unless the context indicates otherwise. As used herein, “micro” refers to micrometer scale dimensions. As used herein “submicron” refers to greater than about 100 nm to less than about 1 micron (μm).

The present invention provides for simple and rapid filtering of biological samples, whereby a sample can be analyzed in the same device or a different device. In one preferred example, a membrane filter that is particularly useful for the filtration of samples comprising viruses along with other biological materials that need be separated from the viruses is used.

### Membrane Filters

Membrane-based technology has been identified as a useful method for the separation of biomaterials including viruses, owing to its efficiency, ease of implementation, and cost effectiveness. Several types of membranes have been employed for virus filtration. For example, microfiltration (MF) membranes show a relatively high flux and good retention of viruses on the membrane due to the presence of electrostatic interactions under appropriate conditions. Ultrafiltration membranes with smaller pore sizes have also been employed for the separation of viruses.

In one embodiment, the viral filters used in accordance with the invention are asymmetric and comprise any one or more of submicron, micron or nano porous polymer membrane structures having a graded porosity, i.e., graded pore size progressing from one major surface of the membrane to the other major surface thereof wherein the pore sizes range from, for example about 10 nm to about 100 microns. Preferably the pore sizes range from about 20 nm to about 100 microns, preferably from about 20 nm to about 50 microns, preferably from about 20 nm to about 10 microns, preferably from about 20 nm to about 1 micron, preferably from about 20 nm to about 0.5 micron, preferably from about 40 nm to about 100 microns, preferably from about 40 nm to about 50 microns, preferably from about 40 nm to about 10 microns, preferably from about 40 nm to about 1 micron, preferably from about 40 nm to about 0.5 microns, preferably from about 80 nm to about 100 micron, preferably from about 80 nm to about 50 microns, preferably from about 80 nm to about 10 microns, preferably from about 80 nm to about 1 micron, preferably from about 80 nm to about 0.5 micron preferably from about 100 nm to about 100 micron, preferably from about 100 nm to about 50 microns, preferably from about 100 nm to about 10 microns, preferably from about 100 nm to about 1 micron, preferably from about 100 nm to about 0.5 micron, preferably from about 200 nm to about 50 microns, preferably from about 200 nm to about 10 microns, preferably from about 200 nm to about 1 micron, preferably from about 200 nm to less than about 1 micron, such as from about 200 nm to about 999 nm. Most preferably, a preferred pore size distribution is in the submicron range.

Depending on production methods for the membranes, the membrane filters may be isoporous, hierarchical, asymmetric graded membranes. An isoporous graded membrane has a surface layer and an asymmetric substructure. The surface layer can have a range of thicknesses. For example, the surface layer can have a thickness of from about 20 nm to about 500 nm preferably about 50 nm to about 300 nm, preferably about 50 nm to 100 nm, including all values to the nm and ranges therebetween. The surface layer has a plurality of pores extending thorough the depth of the surface layer. The pores can have morphologies such as cylindrical and cubic morphologies. The pores can have a size (e.g., diameter) of, for example, from 20 nm to less than about 1 micron such as 100 nm, including all values to the nm and ranges therebetween. At least one surface layer may comprise an ordered array-like porous layer to form a simple sieve.

At least one surface layer can have a range of pore densities. Preferably, the surface layer is isoporous. By “isoporous” it is meant that the pores have narrow pore size distribution. For example, a narrow pore size distribution is less than 0.3 (e.g., 0.1 to 0.3, including all values to 0.01 and ranges therebetween), where the pore size distribution is defined as the coefficient of variance, σ/μ, obtained through a lognormal distribution fit. In various examples, the pore size distribution is 0.1, 0.15, 0.2, 0.25, or 0.3.

The asymmetric substructure may also have a range of thicknesses. For example, the asymmetric substructure layer can have a thickness of from about 20 nm to about 500 nm preferably about 50 nm to about 300 nm, preferably about 50 nm to 100 nm, including all values to the nm and ranges therebetween. The surface layer has a plurality of pores extending thorough the depth of the surface layer. The pores can have morphologies such as cylindrical and cubic morphologies. The pores can have a size (e.g., diameter) of from 40 nm to less than about 1 micron such as between 100 nm and 999 nm including all values to the nm and ranges therebetween.

Polymer materials with continuous (i.e., accessible) hierarchical porosity across multiple length scales ranging from nanometers to micrometers offer the potential for efficient transport of matter through the pores and mechanically robust structures while maintaining ease of processability and relatively high surface areas.

Methods of making of isoporous membranes are known in the art including those that are fabricated from a combination of block copolymer self-assembly and non-solvent induced phase separation (SA+NIPS=SNIPS). The SNIPS derived films are fabricated from chemically distinct block copolymers so that, for example, pore surface chemistries can be tailored via a “mix and match” approach, i.e. the simple blending of the corresponding individual block copolymers into the original polymer solution from which the membrane is cast.

It is believed that the morphology of the surface layer is, in part, a result of the self-assembly of the multiblock copolymer(s) The morphology of this layer is dependent on the casting conditions (e.g., flow rate of environment around the film, water (humidity)/solvent concentration in environment around the film, evaporation time, casting speed, gate height) as well as the composition of the casting solution (e.g., polymer molar mass, chemistry, concentration, casting solvent or mixture of solvents).

Methods for making isoporous membranes may be found in, for example WO2019/023135), WO2019/178045, WO2017/189697, WO 2019/060390, U.S. Pat. Pub. 2017/0327649, and WO2015/048244. In another method termed spinodal-decomposition induced macro- and meso-phase separation plus extraction by rinsing, or SIM²PLE, hierarchical pores are generated by a combination of spinodal decomposition and microphase separation induced via solvent evaporation in a mixture of a block copolymer and a small molar mass additive Dorin et al., Chem. Mater. 2014, 26, 339-347. Other methods for making asymmetric polymeric membranes including track etching ((Lee A, Elam J W, Darling S B (2016) Environ Sci Water Res Technol 2:17-42. doi:10.1039/C5EW00159E; Khulbe K C, Matsuura T, Feng C (2015) TIn: Thakur V K, Thakur M K (eds) Handbook of polymers for pharmaceutical technologies, structure and chemistry. Wiley, New Jersey, United States, pp 33-66), laser ablation (Pazokian H, Jelvani S, Barzin J et al (2011) Opt Commun 284:363-367 doi:10.1016/j.optcom.2010.08.058) and phase inversion such as those described above and in Khorsand-Ghayeni et al., (11 Oct. 2016) Polym. Bull. DOI 10.1007/s00289-01601823-z and Wang Z, Sun L, Wang Q et al (2014) Eur Polym J 60:262-272. doi:10.1016/j.curpolymj.2014.09.015; Barzin J, Madaeni S S, Pourmoghadasi S (2007) J Appl Polym Sci 104:2490-2497. doi:10.1002/app.25627.

In another embodiment, the membrane filter may comprise two different membranes thereby forming a dual layer membrane wherein one layer has a three-dimensional mesh of micron-sized pores capable of acting as a depth filter, and the other membrane has nanometric simple sieves that may be brought together such that they collectively function as a single filter. Each of these membranes may be created by any methods known in the art. For example, a depth filter layer may be produced as described in U.S. Pat. No. 9,333,481.

Examples of a simple sieve layer include but are not limited to: Ulbricht, M., “Advanced functional polymer membranes,” Polymer 47 (2006), pp. 2217-2262.

A preferred dual layer membrane generally includes a first porous layer and a second porous layer adjacent the first porous layer. The first porous layer has a size and characteristics of trapping larger biological components such as whole cells and debris without clogging. The pores of the first porous layer generally have a random orientation and a size greater than 100 nm. The random orientation reduces the tendency of the filter to clog during use. The second porous layer is positioned adjacent the first porous layer and has a smaller pore diameter. generally within the range of 20-100 nm. See Yang et al., 2006 Adv. Mater., 18, 709-712 doi: 10.1002/adma.200501500. The pore size of the second porous layer may be selected to allow a particular virus to pass through the pores. In this embodiment, the membrane material may include a first layer that includes a blood separator, e.g., VF2, GF/DVA, MF1, or Fusion 5, and a second layer that is bonded with the first layer and made to precise dimensions using photolithographic techniques, such as described in U.S. patent application Ser. No. 17/067,528, entitled “Tangential Flow Cassette-HF Emulation” which was filed Oct. 9, 2020, which is incorporated by reference herein. The techniques for making a porous polymeric membrane using techniques adapted from semiconductor manufacturing technology are described in the '528 application. These techniques allow control of the pore size to exact dimensions and may be used to create the second layer of the membrane filter according to an embodiment of the present invention, including a pore size between 20-100 nm, between 50-100 nm or between 80-100 nm as desired.

Preferably the pore sizes range from about 20 nm to about 100 microns, preferably from about 20 nm to about 50 microns, preferably from about 20 nm to about 10 microns, preferably from about 20 nm to about 1 micron, preferably from about 20 nm to about 0.5 micron, preferably from about 40 nm to about 100 microns, preferably from about 40 nm to about 50 microns, preferably from about 40 nm to about 10 microns, preferably from about 40 nm to about 1 micron, preferably from about 40 nm to about 0.5 microns, preferably from about 80 nm to about 100 micron, preferably from about 80 nm to about 50 microns, preferably from about 80 nm to about 10 microns, preferably from about 80 nm to about 1 micron, preferably from about 80 nm to about 0.5 micron preferably from about 100 nm to about 100 micron, preferably from about 100 nm to about 50 microns, preferably from about 100 nm to about 10 microns, preferably from about 100 nm to about 1 micron, preferably from about 100 nm to about 0.5 micron. preferably from about 200 nm to about 50 microns, preferably from about 200 nm to about 10 microns, preferably from about 200 nm to about 1 micron, preferably from about 200 nm to less than about 1 micron, such as from about 200 nm to about 999 nm. Most preferably, a preferred pore size distribution is in the submicron range.

The membrane filters in accordance with the invention may be provided in a sheet form, as a component of a lateral flow device (e.g., immunoassay), or within a syringe filter.

### Lateral Flow Device Application

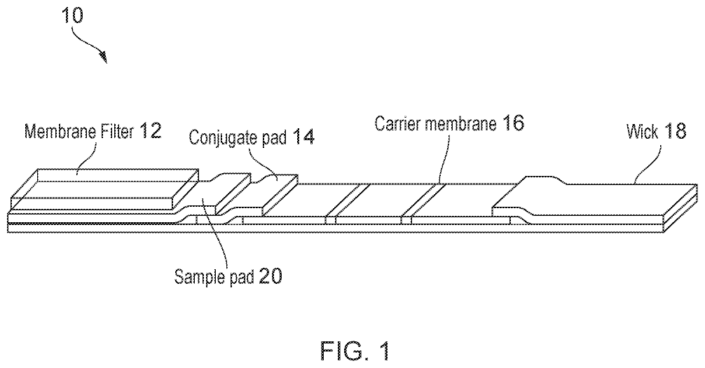

In one aspect, the membrane filter 12 is used in a lateral flow device 10, such as in a lateral flow immunoassay. Preferably, the immunoassay is designed to detect a particular virus that can pass through the membrane filter. The lateral flow device comprises a filter membrane 12, a carrier membrane 16 comprising a test line and a control line, the carrier membrane 16 being in fluid communication with the filter membrane 12; and an absorbent pad (e.g. a wick 18) in fluid communication with the carrier membrane 16, wherein the lateral flow device operates using passive capillary action. The lateral flow device 10 may also comprise a sample pad 20 and/or a conjugate pad 14. The membrane filter is preferably placed in contact with the sample pad or in place of the sample pad within a lateral flow device having the structure of FIG. 1.

The membrane layer 12 is preferably positioned with the smaller pores in a face down orientation in contact with the sample pad, and larger random pores oriented above the portion in contact with the sample pad. Lateral flow devices are intended to detect the presence or absence of a target analyte in a liquid sample. Conventionally, a series of liquid conduits, for example capillary pads, such as pieces of porous paper or sintered polymer are formed on a support. A known arrangement employs various liquid conduit elements, including a first sample liquid receiving element which acts as a sponge and holds an excess of sample liquid. Once soaked, the fluid propagates to a second element, known as a conjugate release pad, in which the manufacturer has stored the so-called conjugate, typically a dried format of bio-active particles in a dissolvable matrix that includes reagents to produce a chemical reaction between the target molecule and its chemical partner that has been immobilized on the particle's surface. As the sample dissolves the particles a reaction takes place to bind the analyte to the particle. Typically, a second reagent, for example a color-changing reagent located at a specific distance along the conjugate pad, or on a third element, and is used to capture particles on which are bound the analyte to provide a test result. A third reagent, for example s color-changing reagent further along the liquid path than the second reagent is often used to capture all particles, and so is used as a control to ensure that the liquid sample has propagated past the second reagent. Examples of lateral flow assays useful in accordance with the invention include those described in U.S. Pat. No. 10,551,381; Yen C. W. et al. Lab Chip. 2015; 15:1638-1641. doi:10.1039/C5LC00055F and Koczula and Gallota, (2016) Essays Biochem; 60(1):111-120 doi:10.1042/EBC20150012; U.S. Pat. App. No. 2017/0115287A1, filed Mar. 17, 2015, entitled “Improvements in and Related to Lateral Flow Testing.”

After passing the reaction zones of the second and third reagents the liquid sample enters the final porous wick material element 18, acting as a waste container.

Diagnostic viral vaccine assays and applications provides many advantages including but not limited to:
Determine treatment strategies
Predict disease course and expected outcome
Predict the potential for virus spread
Allow identification and vaccination of susceptible individuals.
Trace the movement of a virus through world-wide.
   Methods for identifying virus in an infected patient:
ideally should be sensitive, specific, and rapid.
Common targets for diagnostic (Dx)-tests: viral proteins (antigens), viral genomes, and/or antiviral antibodies.
Some designed to detect directly from a patient sample (blood, throat swab) e.g. lateral Dx flow assays
Epidemiologic studies may include large sample cohort requiring use of low cost, high throughput modalities.

The viral filter membranes and the diagnostic tests in accordance with the invention may be used for detection of viruses including but not limited to, SARS-COV-2, SARS (2003), Adenovirus, Norovirus, Rotavirus A, flu (e.g. influenza A), Zika, dengue, chikungunya, West Nile virus, Japanese encephalitis, HIV, H1N1, Epstein Barr virus (EBV), herpes simplex 1 virus (HSV-1), yellow fever virus, ebola virus, Marburg virus, and all variants of the foregoing viruses. The pore sizes of the viral filter membranes of the invention are chosen in order to allow the viral target to be separated from other components of the biological sample. Examples of viral particle sizes which should be taken into account when choosing pore sizes and ranges of specific filter range from about 30 nm, for example which is the particle size of the polio virus, to between about 88-110 nm which is the range of particle sizes for adenovirus and influenza A virus. The particle sizes of HIV-1 virus are in the range of 120 to 150 nm while the HSV-1 particle size is about 125 nm and EBV virus particles around 140 nm.

In a preferred embodiment, the viral filters and diagnostic tests in accordance with the invention are suitable for use in diagnostics tests for the detection in a biological sample of SARS-COV-2 which has a viral particle size in the range of about 70 nm to about 110 nm.

In accordance with the invention, the biological sample is selected from the group comprising any bodily fluid or tissue including but not limited to peripheral blood, sera, plasma, ascites, urine, cerebrospinal fluid (CSF), sputum, saliva, a solid tissue sample, a skin swab sample, a throat swab sample and a genital swab sample.

### Other Viral Filtration Applications

The membrane filters of the invention are useful in other viral filtration settings including but not limited to:
Biopharmaceutical and clinical applications, i.e.:
   Virus clearance in manufacture of therapeutic proteins
   Final purification step in downstream processing of mAb production
   Purification of virus for production of prophylactic vaccines and gene therapy
Monitoring of microbiological water quality
Air purification

### Other Uses of Membrane Filters

The membrane filters of the invention are useful in a variety of particle (both animate and inanimate) separations. Typical separations include eukaryotic and prokaryotic cells (down to virus and fungal spore and seed dimensions) and soil, exhaust emission, metal particulates. The membrane filters of the invention are also suitable for cell-molecule separations.

In order to assess a membrane filter's performance, for example, polystyrene beads or molecules such as poly dextrans of different sizes are used to test filters as a generic test of separation. Most separations are sigmoidal in profile due to the range of sizes in even supposedly uniform sized pore membranes. A key performance “success”, for example, would be a very sharp size cut off profile plus a well-defined pressure—(air & water) flow relationship through the membranes.

In one preferred embodiment, the orientation of the filter of the invention facilitates its use in a specific setting. For example, when a filter is used in connection with a lateral flow assay to detect the presence of a virus, the filter is orientated so that the larger pores of the pore gradient receive the biological sample containing the suspected virus. The virus then passes through the filter and is concentrated prior to passing through to the sample pad of the lateral flow assay. The orientation of the filter is leveraged depending upon what the target biological species is intended to pass through the filter and onto, for example the lateral flow assay strip.

### EXAMPLES

### Example 1—Testing Various Filters for Use in Viral Diagnostic Assays

| | |
|---|---|
| Mock lateral flow testing 1 | To test if the filter material with a nanopore size can be |
| | used in an application where there is no driving force |
| | (e.g. pump) for liquid filtration, except for a passive |
| | capillary action. |
| Sample type | PBS buffer |
| Sample volume | 20 microliters |
| Filter pore size | 20 nm, 50 nm, 100 nm, 150 nm, 200 nm |
| | (configuration: inverted and non-inverted) |
| Number of filters required | 20 replicates |
| Testing method | Build a mock lateral flow test and determine the time the |
| | sample needs to travel onto: the membrane, the test line, |
| | the control line in comparison to the same system |
| | containing no filter. |
| Acceptance criteria | For first test, no acceptance criteria - to generate |
| | performance data first. |

| Mock lateral flow testing 2 | To test if the filter material with a nanopore size can be | |
|---|---|---|
| | used with a very viscous sample in an application where | |
| | there is no driving force (e.g. pump) for liquid filtration, | |
| | except for a passive capillary action. | |
| Sample type | Saliva or other viscous solution. | |
| | Other preferred sample would be whole blood, if | |
| | available. | |
| Sample volume | 100 microliters | |
| Filter pore size | 20 nm, 50 nm, 100 nm, 150 nm, 200 nm (configuration: | |
| | inverted with | |
| | random large pores on top) | |
| Filter format/size | Same as mock testing 1 | |
| Number of filters required | 20 replicates - same as mock testing 1 | |
| Testing method | Same outline as mock testing 1 | |
| Acceptance criteria | Same as mock testing 1 | |
| VFE Testing | Filtration efficiency of viral particles determined by comparing the | |
| | challenge sample collected downstream with the known challenge | |
| | delivered upstream. | |
| Sample type | (1) | Virus spiked buffer |
| | (2) | and/or biological sample (saliva) |
| VFE controls | (3) | Unspiked biological sample as assay control for false positives |
| | (4) | Unspiked clear buffer as assay control for false positives |
| | (5) | Spiked smaller virus than pore size (e.g. 10⁶ |
| | | copies/mL) in clear buffer (optional) |
| | (6) | Virus spiked sample with no filter in the test system to |
| | | determine total challenge delivered to the test filters |

| | |
|---|---|
| Volume range | TBD - Dependent on the filter format/size |
| Virus type | Commercial available heat-inactivated SARS-CoV-2 (ATCC ® VR- |
| | 1986HK ™) or similar size virus if not available/appropriate for |
| | testing |
| Virus size range | 90-140 nm |
| Virus spike load | Range 10⁶-10³ copies/mL - do 3 virus titers |
| Number of filter | 5 replicates |
| replicates/testing condition | |
| Number of filters required | [3 virus titers × 2 tests × 5 replicates] + [3 controls × 5] = 45 filters |
| | for each filter type |
| Virus log reduction value | >6 LRV |
| acceptance criteria | |
| Filter size/format | Syringe format (TBD) |
| Filter Pore size | 20 nm, 50 nm, 100 nm |
| | Use in inverted configuration (random large pores on top) |
| Detection method | Third party testing using their viral clearance standard |
| | procedure |
| | In house standard lateral immunoassay testing for viral proteins |
| | In house RT PCR testing specific for viral RNA/DNA |

The patent and scientific literature referred to herein establishes the knowledge that is available to those with skill in the art. All cited patents and published or unpublished cited patent applications cited herein are incorporated by reference in their entirety. All other published references, documents, manuscripts and scientific literature etc. cited herein are also fully hereby incorporated by reference too.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not necessarily mutually exclusive and that features from the various embodiments may thus be combined in whole or in part in accordance with the invention.

1. A lateral flow device comprising:
a filter membrane, the filter membrane comprising:
a first porous layer, the first porous layer having randomly oriented pores having a first diameter greater than 100 nm;
a second porous layer, the second porous layer being adjacent the first porous layer and having pores with a second diameter in the range of about 20 to less than about 1 micron.-100 nm.
a carrier membrane comprising a test line and a control line, the carrier membrane being in fluid communication with the filter membrane; and
an absorbent pad in fluid communication with the carrier membrane (16), wherein the lateral flow device operates using passive capillary action.

2. The lateral flow device of claim 1, wherein the lateral flow device further comprises a sample pad in fluid communication with the filter membrane and the carrier membrane.

3. The lateral flow device of claim 2, wherein the second porous layer of the filter membrane contacts the sample pad.

4. The lateral flow device of claim 1, wherein the lateral flow device further comprises a conjugate pad in fluid communication with the filter membrane and the carrier membrane.

5. The lateral flow device of claim 1, wherein the lateral flow device further comprises a conjugate pad in fluid communication with the sample pad and the carrier membrane.

6. The lateral flow device of claim 1, wherein the lateral flow device is for an immunoassay.

7. A syringe filter comprising:
a filter housing; and
a filter membrane, the filter membrane comprising:
a first porous layer, the first porous layer having randomly oriented pores having a first diameter greater than 100 microns;
a second porous layer, the second porous layer being adjacent the first porous layer and having pores with a second diameter in the range of about 20 nm to less than about 1 micron.

8. A filter membrane, the filter membrane comprising:
a first porous layer, the first porous layer having randomly oriented pores having a first diameter greater than 100 microns;
a second porous layer, the second porous layer being adjacent the first porous layer and having pores with a second diameter in the range of 20-100 nm about 20 nm to less than about 1 micron.

9. A lateral flow device comprising:
a filter membrane, the filter membrane comprising:
a surface layer and an asymmetric substructure wherein the surface layer has a thickness of 20 nm to 500 nm and the pore sizes range from about 20 nm to about 1 micron and wherein the asymmetric substructure has a thickness of about 20 nm to about 500 nm and has pore size in the range of 100 nm to about 1 micron,
a carrier membrane comprising a test line and a control line, the carrier membrane being in fluid communication with the filter membrane; and
an absorbent pad in fluid communication with the carrier membrane, wherein the lateral flow device operates using passive capillary action.

10. The lateral flow device of claim 9, wherein the lateral flow device further comprises a sample pad in fluid communication with the filter membrane and the carrier membrane.

11. The lateral flow device of claim 10, wherein the second porous layer of the filter membrane contacts the sample pad.

12. The lateral flow device of claim 9, wherein the lateral flow device further comprises a conjugate pad in fluid communication with the filter membrane and the carrier membrane.

13. The lateral flow device of claim 9, wherein the lateral flow device further comprises a conjugate pad in fluid communication with the sample pad and the carrier membrane.

14. The lateral flow device of claim 9, wherein the lateral flow device is for an immunoassay.
